(19) United States
(12) Patent Application Publication
(10) Pub. No.: US 2023/0375806 A1
(43) Pub. Date: Nov. 23, 2023

(54) OPTICAL LENS

(71) Applicant: Rays Optics Inc., Hukou Township (TW)

(72) Inventors: CHING-LUNG LAI, Hukou Township (TW); MENG-WEI LIN, Hukou Township (TW)

(21) Appl. No.: 17/863,781

(22) Filed: Jul. 13, 2022

(30) Foreign Application Priority Data
May 18, 2022 (TW) 111118522

(51) Int. Cl. G02B 13/00; G02B 9/62; G02B 1/04; A61B 3/14; A61B 3/12

(52) U.S. Cl. CPC G02B 13/0045; G02B 9/62; G02B 1/041; A61B 3/14; A61B 3/12

(57) ABSTRACT

An optical lens used for fundus photography includes a first plastic aspheric lens with a positive refractive power, a second plastic aspheric lens, a third plastic aspheric lens, and a fourth plastic aspheric lens with a positive refractive power arranged in order from an object side to an image side. The number of lenses with refractive powers is less than 9, each lens of the optical lens is a singlet lens, a length between respective optical centers of outermost lens surfaces at opposite ends of the optical lens is within a range of 50 mm to 130 mm, and a back focal length is greater than 20 mm.

### BACKGROUND OF THE INVENTION

### a. Field of the Invention

The invention relates to an optical lens, and, more particularly, to a relay lens that can be used with a mobile device.

### b. Description of the Related Art

Nowadays, smartphones can be used for fundus photography to replace traditional ophthalmoscopes. Using a smartphone to take fundus photos may have various advantages. For example, the image pick-up device needs not to connect with a remote computer system, and the captured fundus images can be analyzed in real time. When a smartphone is used for fundus photography, it is necessary to use an adapter with a relay lens inside to match the lens of the smartphone to obtain a complete fundus image. Therefore, it is desirable to provide a relay lens that can be used with various types of mobile devices and may achieve wider viewing angles, lighter weight, lower fabrication costs, and higher imaging quality.

### BRIEF SUMMARY OF THE INVENTION

According to one aspect of the present disclosure, an optical lens used for fundus photography includes a first plastic aspheric lens with a positive refractive power, a second plastic aspheric lens with a refractive power, a third plastic aspheric lens with a refractive power, and a fourth plastic aspheric lens with a positive refractive power arranged in order from an object side to an image side. The first lens and the fourth lens are the outermost lenses at opposite ends of the optical lens. The number of lenses with refractive powers is less than 9, each lens of the optical lens is a singlet lens, a length between respective optical centers of outermost lens surfaces at opposite ends of the optical lens is within a range of 50 mm to 130 mm, and a back focal length is greater than 20 mm.

According to another aspect of the present disclosure, an optical lens includes a first lens, a second lens, a third lens and a fourth lens arranged in order in a direction. The first lens is an aspheric lens with a positive refractive power, the second lens is an aspheric lens with a refractive power, the third lens is an aspheric lens with a refractive power, and the fourth lens is an aspheric lens with a positive refractive power. The number of lenses with refractive powers of the optical lens is less than 9, and a full field of view (FOV) of the optical lens is within a range of 30 degrees to 55 degrees. The optical lens satisfies a condition of 0.14<D1/OAL<0.5, where D1 denotes a lens diameter of the first lens, and OAL denotes a length between respective optical centers of outermost lens surfaces at opposite ends of the optical lens.

According to the above aspects, the optical lens may have at least one of the advantages of light weight, low fabrication costs, wide viewing angles and high resolution relaying images. Therefore, the optical lens is suitable for matching various types of mobile devices complying with their respective specifications to generate high-quality fundus images.

Other objectives, features and advantages of the invention will be further understood from the further technological features disclosed by the embodiments of the invention wherein there are shown and described preferred embodiments of this invention, simply by way of illustration of modes best suited to carry out the invention.

### BRIEF DESCRIPTION OF THE DRAWINGS

FIG. 1 is a schematic diagram showing an image pick-up system using a relay lens with a smartphone according to an embodiment of the invention.

FIG. 2 shows a schematic structure diagram of an optical lens according to a first embodiment of the invention.

FIG. 3 shows a schematic structure diagram of an optical lens according to a second embodiment of the invention.

FIG. 4 shows a schematic structure diagram of an optical lens according to a third embodiment of the invention.

FIG. 5 is an MTF curve diagram of the optical lens shown in FIG. 2 measured at a spatial frequency of 81 lp/mm.

FIG. 6 is an MTF curve diagram of the optical lens shown in FIG. 3 measured at a spatial frequency of 81 lp/mm.

FIG. 7 is an MTF curve diagram of the optical lens shown in FIG. 4 measured at a spatial frequency of 81 lp/mm.

### DETAILED DESCRIPTION OF THE INVENTION

In the following detailed description of the preferred embodiments, directional terminology, such as “top,” “bottom,” “front,” “back,” etc., is used with reference to the orientation of the Figure(s) being described. The components of the invention can be positioned in a number of different orientations. As such, the directional terminology is used for purposes of illustration and is in no way limiting. Further, “First,” “Second,” etc., as used herein, are used as labels for nouns that they precede, and do not imply any type of ordering (e.g., spatial, temporal, logical, etc.).

The term “lens” refers to an element made from a partially or entirely light-transmissive material with optical power. The material commonly includes plastic or glass. The lens may be, a general lens, a prism, an aperture stop, a cylindrical lens, a bi-conical lens, a cylindrical array lens, a wedge lens, a wedge, or a combination of the foregoing elements.

When the optical lens is used for fundus photography, an image side may refer to one side of an optical path comparatively near an external imaging system (such as a smartphone), and an object side may refer to other side of the optical path comparatively near an object (such as human eyes) to be captured.

A certain region of an object side surface (or an image side surface) of a lens may be convex or concave. Herein, a convex or concave region is more outwardly convex or inwardly concave in the direction of an optical axis as compared with other neighboring regions of the object/image side surface.

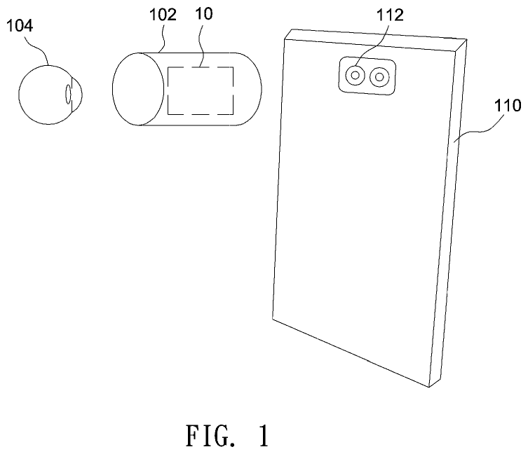

FIG. 1 is a schematic diagram showing an image pick-up system using a relay lens with a smartphone according to an embodiment of the invention. As shown in FIG. 1, a relay lens 10 is accommodated in a casing 102, one end of the casing 102 is attached to a smartphone 110, and the other end of the casing is arranged to point to pupils of human eyes 104. The relay lens 10 performs primary imaging on incoming fundus beams and then converted the primary image into parallel beams, and then the parallel beams are focused by a phone lens 112 of the smart phone 110 to form fundus images, therefore achieving the purpose of capturing fundus images from human eyes 104 by the smart phone 110 and allowing for subsequent processing. It should be noted the smartphone 110 set out in this embodiment is merely an example, and the relay lens 10 can be used with other mobile devices (such as tablet computers) capable of capturing images without limitation.

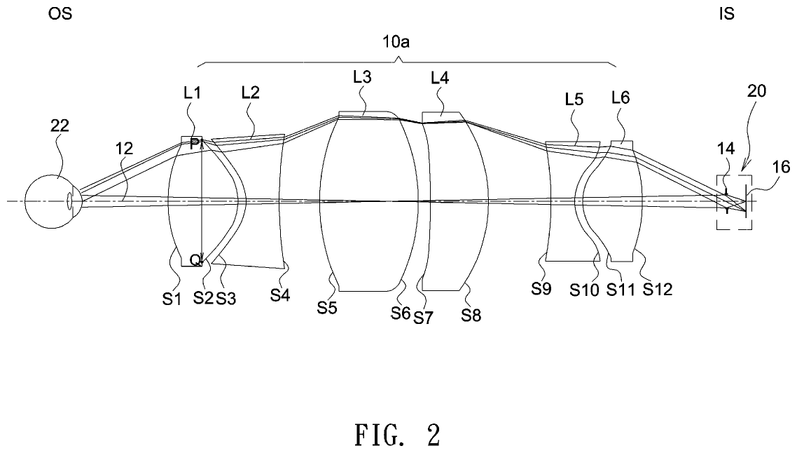

FIG. 2 shows a schematic structure diagram of an optical lens according to a first embodiment of the invention. Referring to FIG. 2, in this embodiment, the optical lens 10a can be used as a relay lens in cooperation with a mobile device to realize fundus photography. For example, the optical lens 10a may relay fundus images of human eyes to a pupil 14 (such as an aperture stop of a phone lens) of an external imaging system 20 (such as a smartphone). The optical lens 10a is accommodated in a casing (not shown) and consists essentially of six lenses with refractive powers including a lens L1, a lens L2, a lens L3, a lens L4, a lens L5 and a lens L6 arranged in order from an object side OS to an image side IS. In this embodiment, the object side OS corresponds to the position of human eyes, and the image side IS corresponds to the position of an image plane 16 of the external imaging system 20 (such as a smartphone). In this embodiment, the refractive powers of the lenses L1-L6 are positive, negative, positive, positive, negative, and positive, respectively, and the lens L1, the lens L2, the lens L3, the lens L4, the lens L5 and the lens L6 are all aspheric lenses made of plastic materials such as PMMA or PC. Furthermore, each of the lenses L1-L6 is a singlet lens, and the lenses L1-L6 can be configured to form a conjugate relationship between pupils of the human eyes 22 and the pupil 14 of the external imaging system 20, so that the optical lens 10a may function as an afocal lens.

According to various embodiments of the invention, the number of lenses with refractive powers is less than 9, but the number, shape and optical characteristic of lenses can be designed according to actual needs without limitation. For example, in one embodiment, the lens L3 can be replaced with two singlet lenses that are stacked together by a spacing of less than 0.05 mm and have substantially the same radius of curvature in two adjacent lens surfaces, and the two singlet lenses may respectively have a high Abbe number and a low Abbe number to facilitate chromatic aberration corrections and hence improve imaging resolution. In other embodiment, in addition to replacing the lens L3 with two singlet lenses as described above, the lens L4 can be also replaced with two singlet lenses having the specific configuration as described above. In other embodiment, the lens L3 can be replaced with three singlet lenses having the specific configuration as described above, and the three singlet lenses may respectively have a high Abbe number, a low Abbe number and a high Abbe number or respectively have a low Abbe number, a high Abbe number and a low Abbe number to facilitate chromatic aberration corrections.

In each of the following embodiments, the object side OS is located on the left side and the image side IS is located on the right side of each figure, and thus this is not repeatedly described in the following for brevity.

Detailed optical data and design parameters of the optical lens 10a are shown in Table 1 below. Note the data provided below are not used for limiting the invention, and those skilled in the art may suitably modify parameters or settings of the following embodiment with reference of the invention without departing from the scope or spirit of the invention.

Table 1 lists the values of parameters for each lens of the optical lens 10a, where the surface symbol denoted by an asterisk is an aspherical surface. Besides, the radius of curvature, thickness/interval and diameter shown in Table 1 are all in a unit of mm.

TABLE 1

| | | | | Re- | |
|---|---|---|---|---|---|
| | | Radius | Interval | fractive | Abbe |
| Object description | Surface | (mm) | (mm) | index | number |
| Eye model | | | 25 | | |
| Lens L1 (aspheric) | S1* | 22.95 | 16.92 | 1.5251 | 56.282 |
| | S2* | −18.73 | 1.71 | | |
| Lens L2 (aspheric) | S3* | −16.47 | 8.30 | 1.5845 | 30.133 |
| | S4* | 157.46 | 9.48 | | |
| Lens L3 (aspheric) | S5* | 57.95 | 24.16 | 1.5845 | 30.133 |
| | S6* | −43.46 | 2.55 | | |
| Lens L4 (aspheric) | S7* | −300.50 | 13.95 | 1.5845 | 30.133 |
| | S8 * | −47.68 | 15.30 | | |
| Lens L5 (aspheric) | S9* | −196.97 | 5.91 | 1.5845 | 30.133 |
| | S10* | 18.35 | 1.77 | | |
| Lens L6 (aspheric) | S11* | 19.35 | 14.16 | 1.5251 | 56.282 |
| | S12* | −25.86 | 20.68 | | |
| Exit pupil 14 | | inf. | 4.74 | | |
| Image plane 16 | | inf. | 0.00 | | |

In the above Table 1, the field heading “interval” represents a distance between two adjacent surfaces along the optical axis 12 of the optical lens 10a. For example, an interval of the surface S1 is a distance between the surface S1 and the surface S2 along the optical axis 12. Further, the interval, refractive index and Abbe number of any lens listed in the column of “Object description” show values in a horizontal row aligned with the position of that lens. Moreover, in table 1, the surfaces S1 and S2 are respectively the object-side surface and image-side surface of the lens L1, the surfaces S3 and S4 are respectively the object-side surface and image-side surface of the lens L2, and the remaining lens surfaces are classified by analogy so that related descriptions are omitted for sake of brevity.

The radius (radius of curvature) in the above table is a reciprocal of the curvature. When a lens surface has a positive radius of curvature, the center of the lens surface is located towards the image side. When a lens surface has a negative radius of curvature, the center of the lens surface is located towards the object side.

According to various embodiments of the invention, a back focal length (a distance between the lens surface closest to the image side IS and the pupil 14 of the external imaging system) may be greater than 20 mm, and an overall length OAL may be within a range of 50 mm to 130 mm, preferably 55 mm to 128 mm, and more preferably 60 mm to 126 mm. Herein, two outermost lens surfaces (such as the surfaces S1 and S12 shown in FIG. 2) at opposite ends of the optical lens 10a respectively have a first optical center and a second optical center, and the overall length OAL is defined as the distance between the first optical center and the second optical center. Furthermore, in one embodiment, the optical lens can be adapted to an external lens system (such as a smartphone lens), a first distance is measured from a lens surface of the optical lens closest to a subject to be captured (such as human eyes) to the subject, a second distance is measured from a lens surface of the optical lens closest to the external lens system to an aperture stop of the external lens system, and a ratio of the first distance to the second distance may be within a range of 0.95 to 1.05.

In this embodiment, a back focal length of the optical lens 10a is 20.68 mm, an overall length OAL is 114.206 mm, an effective focal length EFL is 199.609 mm, an object distance (a distance between an eye model 22 and a lens surface S1 closest to the object side OS) is 25 mm, a diameter of an entrance pupil (a pupil of the eye model) is 4 mm, and a diameter of the exit pupil 14 (an aperture stop of the external imaging system) is 2.648 mm.

FOV stands for a light collection angle of an optical surface S1 closest to the magnified side OS; that is, the FOV is a full field of view measured by a horizontal line and a vertical line. According to an embodiment of the invention, the FOV is within a range of 30 degrees to 55 degrees, preferably 32 degrees to 54 degrees, and more preferably 34 degrees to 53 degrees. In this embodiment, the full field of view FOV of the optical lens 10a is 44.189 degrees.

Each of the lenses may be assigned a parameter of “lens diameter”. For example, as shown in FIG. 2, the lens L1 has an object-side surface S1 and an image-side surface S2, the light-transmitting area of the lens surface provides two turning points P and Q at opposite ends of the optical axis 12, and a lens diameter is the maximum distance between the turning points P and Q measured in the direction perpendicular to the optical axis 12. In this embodiment, a distance between the turning points P and Q on the image-side surface S2 is greater than a distance between the turning points P and Q on the object-side surface S1. Therefore, a lens diameter D1 of the lens L1 is the distance between the turning points P and Q on the image-side surface S2 in the direction perpendicular to the optical axis 12. In this embodiment, the diameter D1 of the lens L1 is 28.51 mm. Furthermore, according to various embodiments of the invention, the optical lens may satisfy the condition of 0.14<D1/OAL<0.5, preferably satisfy the condition of 0.15<D1/OAL<0.46, and more preferably satisfy the condition of 0.16<D1/OAL<0.42, where D1 is the lens diameter of the lens L1 closest to the object side, and OAL is the overall length. Meeting the above condition is beneficial to properly converge the captured beams entering the relay optical lens to allow for better optical performance in a limited space. Furthermore, in this embodiment, the two outermost lenses (lens L1 and lens L6) of the optical lens 10a have the two smallest lens diameters among all lenses of the optical lens 10a. In addition, in this embodiment, the lens L3 and the lens L4 have the two largest lens diameters among all lenses of the optical lens 10a.

An aspheric lens indicates at least one of its front lens surface and rear lens surface has a radius of curvature that varies along a center axis to correct abbreviations. In the following design examples of the invention, each aspheric surface satisfies the following equation:

$$Z = \frac{{cr}^{2}}{1 + \sqrt{1 - ( 1 + k ) {c}^{2} {r}^{2}}} + {Ar}^{4} + {Br}^{6} + {Cr}^{8} + {Dr}^{10} + {Er}^{12} + {Fr}^{14} + … ,$$

where Z denotes a sag of an aspheric surface along the optical axis 12, c denotes a reciprocal of a radius of an osculating sphere, K denotes a Conic constant, r denotes a height of the aspheric surface measured in a direction perpendicular to the optical axis 12, and parameters A-I listed in Table 2 are 4th, 6th, 8th, 10th, 12th, 14th, 16th, 18th and 20th order aspheric coefficients. Note the data provided below are not used for limiting the invention, and those skilled in the art may suitably modify parameters or settings of the following embodiment with reference of the invention without departing from the scope or spirit of the invention.

TABLE 2

| Surface | S1* | S2* | S3* | S4* | S53 | S6* |
|---|---|---|---|---|---|---|
| K | 0 | 0 | 0 | 0 | 0 | 0 |
| A | −1.38E−04 | −6.22E−04 | −1.29E−04 | 5.13E−04 | 2.22E−05 | −1.87E−05 |
| B | 2.41E−06 | 9.24E−06 | −1.07E−05 | −1.63E−05 | −5.63E−07 | 1.76E−07 |
| C | −2.75E−08 | −4.84E−08 | 3.35E−07 | 2.52E−07 | 7.93E−09 | −2.42E−09 |
| D | 2.06E−10 | −1.29E−10 | −4.54E−09 | −2.48E−09 | −6.28E−11 | 3.62E−11 |
| E | −1.21E−12 | 3.31E−12 | 3.63E−11 | 1.69E−11 | 3.00E−13 | −2.88E−13 |
| F | 5.74E−15 | −2.06E−14 | −1.82E−13 | −7.87E−14 | −8.85E−16 | 1.26E−15 |
| G | −2.05E−17 | 6.59E−17 | 5.70E−16 | 2.40E−16 | 1.58E−18 | −3.11E−18 |
| H | 4.54E−20 | −1.12E−19 | −1.02E−18 | −4.27E−19 | −1.58E−21 | 4.11E−21 |
| I | −4.45E−23 | 8.12E−23 | 8.02E−22 | 3.35E−22 | 6.71E−25 | −2.26E−24 |
| Surface | S7* | S8* | S9* | S10* | S11* | S12* |
| K | 0 | 0 | 0 | 0 | 0 | 0 |
| A | 1.58E−05 | −2.00E−05 | −5.19E−04 | 1.21E−04 | 6.25E−04 | 1.34E−04 |
| B | −1.80E−07 | 5.65E−07 | 1.63E−05 | 1.07E−05 | −9.25E−06 | −2.40E−06 |
| C | 2.42E−09 | −7.93E−09 | −2.52E−07 | −3.35E−07 | 4.83E−08 | 2.76E−08 |
| D | −3.62E−11 | 6.28E−11 | 2.48E−09 | 4.54E−09 | 1.29E−10 | −2.06E−10 |
| E | 2.88E−13 | −3.00E−13 | −1.69E−11 | −3.63E−11 | −3.31E−12 | 1.21E−12 |
| F | −1.26E−15 | 8.85E−16 | 7.87E−14 | 1.82E−13 | 2.06E−14 | −5.75E−15 |
| G | 3.11E−18 | −1.58E−18 | −2.40E−16 | −5.70E−16 | −6.59E−17 | 2.05E−17 |
| H | −4.11E−21 | 1.58E−21 | 4.27E−19 | 1.02E−18 | 1.12E−19 | −4.52E−20 |
| I | 2.26E−24 | −6.73E−25 | −3.35E−22 | −8.01E−22 | −8.12E−23 | 4.50E−23 |

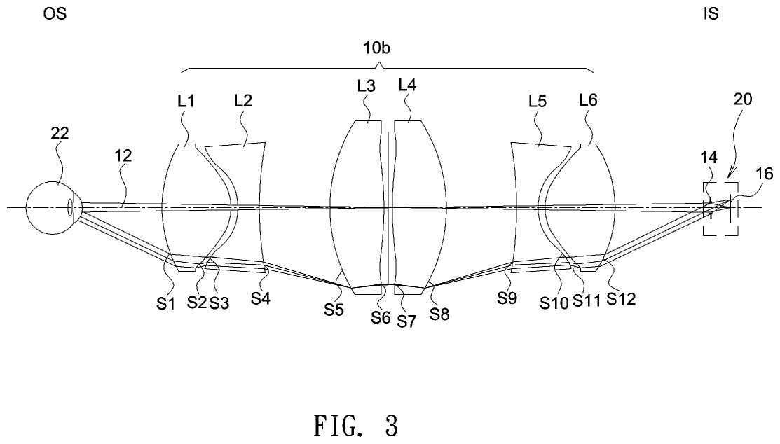

FIG. 3 shows a schematic structure diagram of an optical lens 10b according to a second embodiment of the invention. Referring to FIG. 3, the optical lens 10b includes a lens L1, a lens L2, a lens L3, a lens L4, a lens L5 and a lens L6 in order from the object side OS to the image side IS, and refractive powers of the lenses L1-L6 are respectively positive, negative, positive, positive, negative and positive. In this embodiment, the lens L1, the lens L2, the lens L3, the lens L4, the lens L5, and the lens L6 are all plastic aspheric lenses. Each of the lenses L1-L6 is a singlet lens, the lenses L1-L6 are substantially configured in bilateral symmetry, and the optical lens 10b is configured as an afocal lens, but the invention is not limited thereto. In this embodiment, an object distance is 25 mm, and a back focal length is 25 mm; that is, a distance between the entrance pupil (such as the pupil of the eye model) and its nearest lens surface (surface S1) is substantially equal to a distance between the exit pupil (such as an aperture stop of a smartphone) and its nearest lens surface (surface S12). In this embodiment, an effective focal length EFL of the optical lens 10b is −36290.5 mm, an overall length OAL is 117.243 mm, a diameter of the entrance pupil is 4 mm, a diameter of the exit pupil is 2.648 mm, an FOV is 50 degree, and a lens diameter D1 of the lens L1 is 29.692 mm. Detailed optical data and design parameters of the optical lens 10b are shown in Table 3, and table 4 lists aspheric coefficients and conic constant of each aspheric surface of the optical lens 10b.

TABLE 3

| | | | | Re- | |
|---|---|---|---|---|---|
| | | Radius | Interval | fractive | Abbe |
| Object description | Surface | (mm) | (mm) | index | number |
| Eye model | | | 25 | | |
| Lens L1 (aspheric) | S1* | 21.71 | 17.56 | 1.5251 | 56.282 |
| | S2* | −19.10 | 1.90 | | |
| Lens L2 (aspheric) | S3* | −16.09 | 6.25 | 1.5845 | 30.133 |
| | S4* | 186.79 | 18.02 | | |
| Lens L3 (aspheric) | S5* | 51.63 | 13.69 | 1.5845 | 30.133 |
| | S6* | −120.02 | 2.38 | | |
| Lens L4 (aspheric) | S7* | 120.02 | 13.69 | 1.5845 | 30.133 |
| | S8 * | −51.63 | 18.02 | | |
| Lens L5 (aspheric) | S9* | −186.79 | 6.25 | 1.5845 | 30.133 |
| | S10* | 16.09 | 1.90 | | |
| Lens L6 (aspheric) | S11* | 19.10 | 17.56 | 1.5251 | 56.282 |
| | S12* | −21.71 | 25.00 | | |
| Exit pupil 14 | S13* | inf. | 4.74 | | |
| Image plane 16 | S14* | inf. | 0.00 | | |

TABLE 4

| Surface | S1* | S2* | S3* | S4* | S5* | S6* |
|---|---|---|---|---|---|---|
| K | 0 | 0 | 0 | 0 | 0 | 0 |
| A | −1.39E−04 | −6.22E−04 | −1.30E−04 | 5.13E−04 | 2.04E−05 | −1.58E−05 |
| B | 2.42E−06 | 9.24E−06 | −1.07E−05 | −1.63E−05 | −5.64E−07 | 1.79E−07 |
| C | −2.75E−08 | −4.84E−08 | 3.35E−07 | 2.52E−07 | 7.93E−09 | −2.42E−09 |
| D | 2.07E−10 | −1.29E−10 | −4.54E−09 | −2.48E−09 | −6.28E−11 | 3.62E−11 |
| E | −1.21E−12 | 3.31E−12 | 3.63E−11 | 1.69E−11 | 3.00E−13 | −2.88E−13 |
| F | 5.74E−15 | −2.06E−14 | −1.82E−13 | −7.87E−14 | −8.85E−16 | 1.26E−15 |
| G | −2.05E−17 | 6.59E−17 | 5.70E−16 | 2.40E−16 | 1.58E−18 | −3.11E−18 |
| H | 4.54E−20 | −1.12E−19 | −1.02E−18 | −4.27E−19 | −1.58E−21 | 4.11E−21 |
| I | −4.44E−23 | 8.12E−23 | 8.02E−22 | 3.35E−22 | 6.73E−25 | −2.26E−24 |
| Surface | S7* | S8* | S9* | S10* | S11* | S12* |
| K | 0 | 0 | 0 | 0 | 0 | 0 |
| A | 1.58E−05 | −2.04E−05 | −5.13E−04 | 1.30E−04 | 6.22E−04 | 1.39E−04 |
| B | −1.79E−07 | 5.64E−07 | 1.63E−05 | 1.07E−05 | −9.24E−06 | −2.42E−06 |
| C | 2.42E−09 | −7.93E−09 | −2.52E−07 | −3.35E−07 | 4.84E−08 | 2.75E−08 |
| D | −3.62E−11 | 6.28E−11 | 2.48E−09 | 4.54E−09 | 1.29E−10 | −2.07E−10 |
| E | 2.88E−13 | −3.00E−13 | −1.69E−11 | −3.63E−11 | −3.31E−12 | 1.21E−12 |
| F | −1.26E−15 | 8.85E−16 | 7.87E−14 | 1.82E−13 | 2.06E−14 | −5.74E−15 |
| G | 3.11E−18 | −1.58E−18 | −2.40E−16 | −5.70E−16 | −6.59E−17 | 2.05E−17 |
| H | −4.11E−21 | 1.58E−21 | 4.27E−19 | 1.02E−18 | 1.12E−19 | −4.54E−20 |
| I | 2.26E−24 | −6.73E−25 | −3.35E−22 | −8.02E−22 | −8.12E−23 | 4.44E−23 |

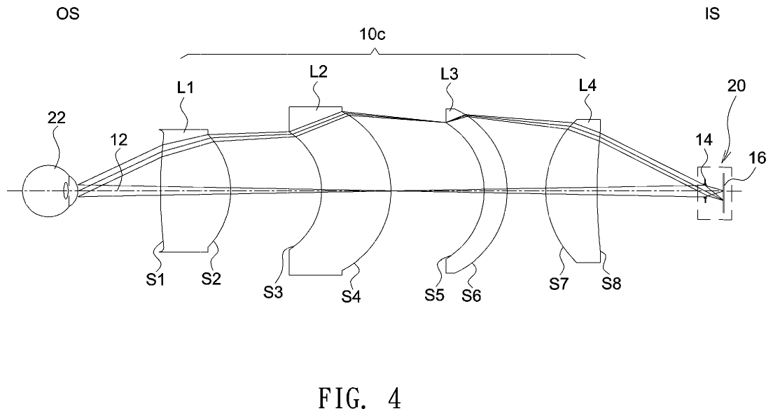

FIG. 4 shows a schematic structure diagram of an optical lens 10c according to a third embodiment of the invention. Referring to FIG. 4, the optical lens 10c includes a lens L1, a lens L2, a lens L3 and a lens L4 in order from the object side OS to the image side IS, and refractive powers of the lenses L1-L4 are respectively positive, positive, negative and positive. In this embodiment, the lens L1, the lens L2, the lens L3 and the lens L4 are all plastic aspheric lenses. Each of the lenses L1-L4 is a singlet lens, and the optical lens 10c is configured as an afocal lens, but the invention is not limited thereto. In one embodiment, the lens L1 can be replaced with two singlet lenses that are stacked together by a spacing of less than 0.05 mm and have substantially the same radius of curvature in two adjacent lens surfaces, and the two single lenses may respectively have a high Abbe number and a low Abbe number to facilitate chromatic aberration corrections and hence improve imaging resolution.

In the embodiment of FIG. 4, an object distance of the optical lens 10c is 25 mm, a back focal length is 30.77 mm, an effective focal length EFL is 8096.386 mm, a total lens length OAL is 124.195 mm, a diameter of the entrance pupil is 4 mm, a diameter of the exit pupil is 2.648 mm, an FOV is 50 degrees, and a lens diameter D1 of the lens L1 is 32.038 mm. Detailed optical data and design parameters of the optical lens 10c are shown in Table 5, and table 6 lists aspheric coefficients and conic constant of each aspheric surface of the optical lens 10c.

TABLE 5

| | | | | Re- | |
|---|---|---|---|---|---|
| | | Radius | Interval | fractive | Abbe |
| Object description | Surface | (mm) | (mm) | index | number |
| Eye model | | | 25 | | |
| Lens L1 (aspheric) | S1* | 179.96 | 20.00 | 1.535 | 55.711 |
| | S2* | −19.83 | 25.76 | | |
| Lens L2 (aspheric) | S3* | −17.78 | 19.69 | 1.5855 | 29.909 |
| | S4* | −24.87 | 26.50 | | |
| Lens L3 (aspheric) | S5* | −21.74 | 6.24 | 1.5855 | 29.909 |
| | S6* | −24.55 | 11.37 | | |
| Lens L4 (aspheric) | S7* | 24.42 | 14.64 | 1.535 | 55.711 |
| | S8* | 217.18 | 30.77 | | |
| Exit pupil 14 | | inf. | 4.74 | | |
| Image plane 16 | | inf. | 0.00 | | |

TABLE 6

| Surface | S1* | S2* | S3* | S4* |
|---|---|---|---|---|
| K | 0 | 0 | 0.047009615 | −0.03370624 |
| A | −7.07E−06 | 1.46E−05 | −9.62E−07 | −6.32E−06 |
| B | −1.09E−08 | 1.23E−08 | 1.13E−08 | −9.91E−10 |
| C | 1.14E−10 | 4.83E−11 | 5.37E−11 | 2.45E−12 |
| D | 5.70E−13 | 1.29E−13 | 2.25E−13 | 8.04E−15 |
| E | 2.25E−15 | 3.38E−16 | 7.58E−16 | 2.13E−17 |
| F | 6.92E−18 | 6.29E−19 | 2.37E−18 | 5.21E−20 |
| G | −8.59E−20 | 3.10E−21 | 7.39E−21 | 9.45E−23 |
| H | 0.00E+00 | 0.00E+00 | 0.00E+00 | 0.00E+00 |
| I | 0.00E+00 | 0.00E+00 | 0.00E+00 | 0.00E+00 |
| Surface | S5* | S6* | S7* | S8* |
| K | 0.032253936 | −0.003635534 | 0 | 0 |
| A | −1.34E−06 | 2.09E−06 | −7.15E−06 | 1.02E−07 |
| B | 7.99E−09 | −4.03E−10 | −8.68E−10 | 3.05E−09 |
| C | 2.09E−11 | −7.86E−13 | −5.45E−12 | 2.02E−11 |
| D | 3.20E−14 | 4.36E−15 | −2.04E−14 | 1.63E−14 |
| E | 1.18E−17 | 2.17E−17 | −4.05E−17 | −1.90E−16 |
| F | −8.73E−20 | 3.89E−20 | −5.06E−21 | −9.73E−19 |
| G | 3.24E−23 | −4.37E−23 | 4.60E−22 | 4.71E−21 |
| H | 0.00E+00 | 0.00E+00 | 0.00E+00 | 0.00E+00 |
| I | 0.00E+00 | 0.00E+00 | 0.00E+00 | 0.00E+00 |

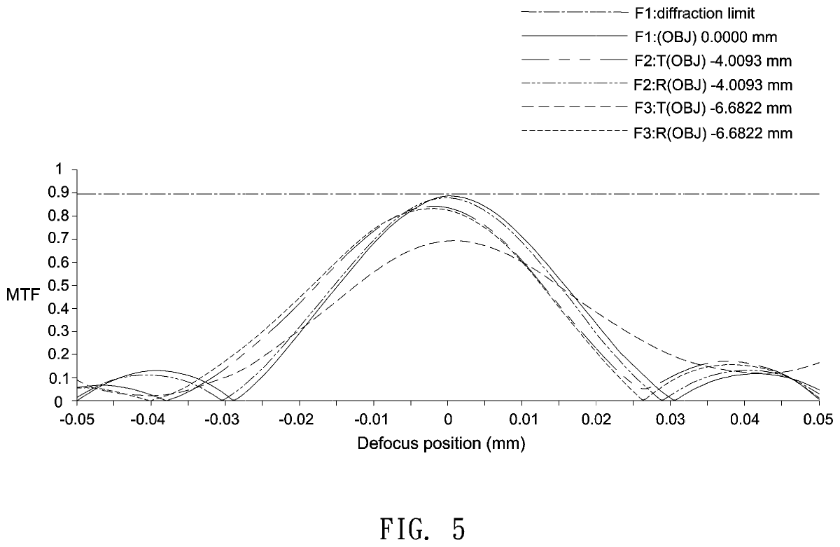

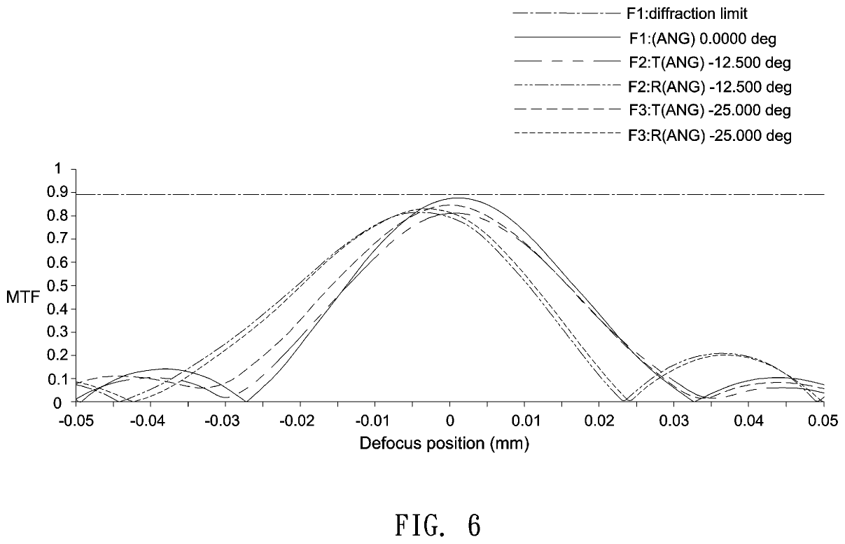

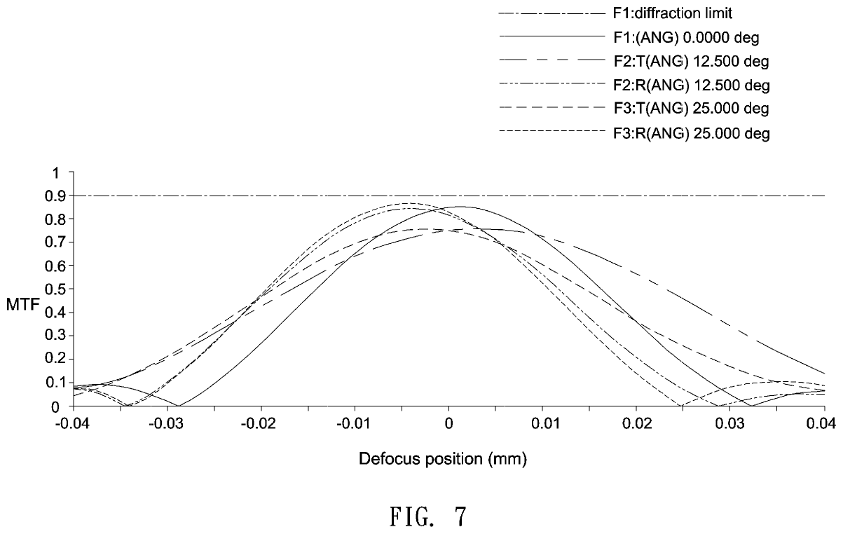

FIG. 5 is a modulation transfer function (MTF) curve diagram of the optical lens 10a shown in FIG. 2 measured at a spatial frequency of 81 lp/mm, FIG. 6 is an MTF curve diagram of the optical lens 10b shown in FIG. 3 measured at a spatial frequency of 81 lp/mm, and FIG. 7 is an MTF curve diagram of the optical lens 10c shown in FIG. 4 measured at a spatial frequency of 81 lp/mm. Because the optical lenses 10a-10c are relay lenses that cannot directly form a final image, the simulation results shown in FIG. 5-7 are realized by the optical lenses 10a-10c cooperating with an ideal lens (a lens without aberrations) having a focal length of 4.74 mm for imaging to verify performance of the optical lenses 10a-10c. The simulation results shown in FIGS. 5-7 are within permitted ranges specified by the standard, which indicates the optical lens 10a-10c can obtain high-resolution relaying images.

According to various embodiments of the invention, by using a large number of plastic aspheric lenses, fabrication costs can be reduced without lowering imaging qualities. In addition, the optical lens may consist essentially of less than 9 lenses and does not have any cemented lens, this may help to reduce fabrication costs and overall weight. Through the design of the various embodiments, the optical lens may have at least one of the following advantages: light weight, low fabrication costs, wide viewing angles and high-resolution relaying images. Therefore, the optical lens is suitable for matching various types of mobile devices complying with their respective specifications to generate high-quality fundus images.

Though the embodiments of the invention have been presented for purposes of illustration and description, they are not intended to be exhaustive or to limit the invention. Accordingly, many modifications and variations without departing from the spirit of the invention or essential characteristics thereof will be apparent to practitioners skilled in this art. It is intended that the scope of the invention be defined by the claims appended hereto and their equivalents in which all terms are meant in their broadest reasonable sense unless otherwise indicated.

What is claimed is:

1. An optical lens used for fundus photography, comprising:
a first plastic aspheric lens with a positive refractive power, a second plastic aspheric lens with a refractive power, a third plastic aspheric lens with a refractive power, and a fourth plastic aspheric lens with a positive refractive power arranged in order from an object side to an image side, and the first lens and the fourth lens being outermost lenses at opposite ends of the optical lens;
wherein a number of lenses with refractive powers of the optical lens is less than 9, each lens of the optical lens is a singlet lens, a length between respective optical centers of outermost lens surfaces at opposite ends of the optical lens is within a range of 50 mm to 130 mm, and a back focal length is greater than 20 mm.

2. The optical lens as claimed in claim 1, wherein the optical lens is an afocal lens.

3. The optical lens as claimed in claim 1, wherein the optical lens has a bilateral symmetry configuration.

4. The optical lens as claimed in claim 1, further comprising:
a fifth lens with a negative refractive power disposed between the first lens and the second lens; and
a sixth lens with a negative refractive power disposed between the third lens and the fourth lens.

5. The optical lens as claimed in claim 1, wherein the two outermost lenses at opposite ends of the optical lens have two smallest lens diameters among all lenses of the optical lens.

6. The optical lens as claimed in claim 1, wherein the second lens and the third lens have two largest lens diameters among all lenses of the optical lens.

7. The optical lens as claimed in claim 1, wherein a distance between an entrance pupil and a lens surface closest to the entrance pupil is substantially equal to a distance between an exit pupil and a lens surface closest to the exit pupil.

8. The optical lens as claimed in claim 1, wherein the optical lens is adapted to an external lens system, a first distance is measured from a subject to be captured to a lens surface of the optical lens closest to the subject, a second distance is measured from an aperture stop of the external lens system to a lens surface of the optical lens closest to the external lens system, and a ratio of the first distance to the second distance is within a range of 0.95 to 1.05.

9. The optical lens as claimed in claim 1, wherein a full field of view (FOV) of the optical lens is within a range of 30 degrees to 55 degrees.

10. The optical lens as claimed in claim 1, wherein the optical lens satisfies one of the following conditions: (1) the optical lens has six lenses with refractive powers of positive, negative, positive, positive, negative and positive respectively in order from the object side to the image side, (2) the optical lens has four lenses with refractive powers of positive, positive, negative and positive respectively in order from the object side to the image side.

11. An optical lens, comprising:
a first lens, a second lens, a third lens and a fourth lens arranged in order in a direction, the first lens being an aspheric lens with a positive refractive power, the second lens being an aspheric lens with a refractive power, the third lens being an aspheric lens with a refractive power, and the fourth lens being an aspheric lens with a positive refractive power, a number of lenses with refractive powers of the optical lens being less than 9, a full field of view (FOV) of the optical lens being within a range of 30 degrees to 55 degrees, and the optical lens satisfying the following condition:
14<D1/OAL<0.5, where D1 denotes a lens diameter of the first lens, and OAL denotes a length between respective optical centers of outermost lens surfaces at opposite ends of the optical lens.

12. The optical lens as claimed in claim 11, wherein a back focal length of the optical lens is greater than 20 mm.

13. The optical lens as claimed in claim 11, wherein the optical lens is an afocal lens.

14. The optical lens as claimed in claim 11, wherein the optical lens has a bilateral symmetry configuration.

15. The optical lens as claimed in claim 11, further comprising:
a fifth lens with a negative refractive power disposed between the first lens and the second lens; and
a sixth lens with a negative refractive power disposed between the third lens and the fourth lens.

16. The optical lens as claimed in claim 11, wherein two outermost lenses at opposite ends of the optical lens have two smallest lens diameters among all lenses of the optical lens.

17. The optical lens as claimed in claim 11, wherein the second lens and the third lens have two largest lens diameters among all lenses of the optical lens.

18. The optical lens as claimed in claim 11, wherein a distance between an entrance pupil and a lens surface closest to the entrance pupil is substantially equal to a distance between an exit pupil and a lens surface closest to the exit pupil.

19. The optical lens as claimed in claim 11, wherein the optical lens is adapted to an external lens system, a first distance is measured from a subject to be captured to a lens surface of the optical lens closest to the subject, a second distance is measured from an aperture stop of the external lens system to a lens surface of the optical lens closest to the external lens system, and a ratio of the first distance to the second distance is within a range of 0.95 to 1.05.

20. The optical lens as claimed in claim 11, wherein the optical lens satisfies one of the following conditions: (1) the optical lens has six lenses with refractive powers of positive, negative, positive, positive, negative and positive respectively in order from the direction, (2) the optical lens has four lenses with refractive powers of positive, positive, negative and positive respectively in order from the direction.
